(19) United States
(12) Patent Application Publication
(10) Pub. No.: US 2012/0141602 A1
(43) Pub. Date: Jun. 7, 2012

(54) SYSTEMS CONTAINING MAGNETIC NANOPARTICLES AND POLYMERS, SUCH AS NANOCOMPOSITES AND FERROFLUIDS, AND APPLICATIONS THEREOF

(75) Inventors: Angel Millan Escolano, Madrid (ES); Fernando Palacio Parada, Madrid (ES); Gemma Ibarz Ric, Madrid (ES); Eva Natividad Blanco, Madrid (ES)

(73) Assignee: Consejo Superior de Investigaciones Cientificas, Madrid (ES)

(21) Appl. No.: 12/442,306

(22) PCT Filed: Aug. 10, 2007
(86) PCT No.: PCT/EP07/58312

§ 371 Date: Sep. 16, 2009

(30) Foreign Application Priority Data
Sep. 22, 2006 (ES) P200602404

(51) Int. Cl. H01F 1/44; C02F 1/48; B03C 1/00; A61K 33/26; A61P 35/00; A61P 37/00; A61P 31/00; C12Q 1/70; C12Q 1/68; C12Q 1/04; F16J 15/02; H01F 1/42; B82Y 30/00; B82B 3/00; B82Y 5/00; B82Y 15/00

(52) U.S. Cl. 424/647; 428/402; 210/695; 252/62.54; 435/5; 435/6.11; 435/34; 277/312; 977/773; 977/779; 977/896; 977/915; 977/920; 977/932

(57) ABSTRACT

The present invention relates to a system comprising magnetic nanoparticles of a metal oxide and a polymer, which in turn contains monomers with different functional groups. This system can be solid (nanocomposite) or liquid (ferrofluid). The present invention also relates to a process for obtaining the system, as well as its use, mainly in biotechnological, veterinary and medical applications, such as, for example, for the diagnosis and treatment of human diseases.

### FIELD OF THE ART

The present invention is comprised within the field of new materials, particularly nanoparticle systems with magnetic properties. It is specifically aimed at systems comprising particles of a metal oxide, comprising iron, and an organic polymer, as well a process for obtaining them and their applications in different fields, including biotechnology and particularly biomedicine.

### STATE OF THE ART

The applications of iron oxide and magnetic ferrite nanoparticles, in solid form such as nanocomposite, or in ferrofluid form, have extended to many areas in industry, and particularly in pharmacy, biochemistry and medicine for several decades [Popplewell J Phys. Technol. 1984, 15, 150]. Their first application in the latter field was as contrast agents in magnetic resonance imaging (MRI) around the 80s [Weissleder, R.; Papisov, M. Rev. Magn. Reson. Med. 1992, 4, 1]. Since then, a great variety of utilities of these nanoparticles in this area have been described [Moghimi, S. M.; Hunter A. C.; Murray, J. C. Pharmacol. Rev. 2001, 53, 283] such as the directed administration of medicinal products [Brigger, I.; Dubernet, C.; Couvreur, P. Adv. Drug. Deliver. Rev. 2002, 54, 631], immunoassays [Lange, J.; Kötitz, R.; Haller, A.; Trahms, L.; Semmler, W.; Weitschies, W. J: Magn. Magn. Mater. 2002, 252, 381], molecular biology [Bogoyevitch, M. A.; Kendrick, T. S.; Ng, D. C. H.; Barr, R. K. DNA Cell Biol. 2002, 21, 879]), DNA nucleic acid purification [Uhlen, M. Nature 1989, 340, 733], cell separation [Safarik, I.; Safarikova, M. J. Chromatogr. B 1999, 722, 33], therapy by means of hyperthermia [Jordan, A.; Scholz, R.; Mater-Hauff, K.; Johannsen, M.; Wust, P.; Nadobny, J.; Schirra, H.; Schmidt, H.; Deger, S.; Loening, S. J. Magn. Magn. Mater., 2001, 225, 118], and others. As regards their industrial applications, their usefulness in magnetic recording [Veitch, R. J. IEEE Trans Magn 2001, 37, 1609], magnetic refrigeration [Bisio, G.; Rubatto, G.; Schiapparelli, P. Energ. Conyers. Manage. 1999, 40, 1267], magnetic printing [Meisen, U.; Kathrein, H. J. Imaging Sci. Techn 2000, 44, 508], magnetic inks [Manciu, F. S., Manciu, M.; Sen, S. J. Magn. Magn. Mater. 2000, 220, 285], lubrication and sealing in vacuum systems [Bhimani, Z.; Wilson, B. Ind. Lubr. Tribol. 1997, 49, 288], damping systems [Kamiyama, S.; Okamoto, K.; Oyama, T. Energ. Conyers. Manage. 2002, 43, 281], magnetic sensors [Crainic, M. S.; Schlett, Z. J. Magn. Magn. Mater. 2004, 268, 8], actuators [Buioca, C. D.; Iusan, V.; Stanci, A.; Zoller, C. J. Magn. Magn. Mater. 2002, 252, 318], catalysis [Liao, M. H.; Chen, D. H. J. Mol. Catal. B-Enzym. 2002, 18, 81.], metal recovery and water purification [Takafuji, M.; Ide, S.; Ihara, H.; Xu, Z. H. Chem. Mater. 2004, 16, 1977], magnetic membranes [Sourty, E.; Ryan, D. H.; Marchessault, H. Cellulose 1998, 5, 5], inductors and antenas in communications technology [Korenivski, V. J. Magn. Magn. Mater. 2000, 215, 800], magnetic shields and microwave absorption [Rozanov, K. N. IEEE Trans. Ant. Propagat. 2000, 48, 1230], smart materials [Chatterjee, J.; Haik, Y.; Chen, C. J. Colloid Polym. Sci. 2003, 281, 892], magneto-conductive materials [Sunderland, K.; Brunetti, P.; Spinu, L.; Fang, J. Y.; Wang, Z. J.; Lu, W. G. Mater. Lett. 2004, 58, 3136], transparent magnets [Vassiliou, J. K.; Mehrotra, V.; Otto, J. W.; Dollahon, N. R. Mater. Sci. Forum 1996, 225, 725], luminescent magnets [Wang, D. S.; He, J. B.; Rosenzweig, N.; and Rosenzweig, Z. Nano Lett. 2004, 4, 409], magneto-optical devices [Redl, F. X.; Cho, K. S.; Murray, C. B.; O'Brien, C. B. Nature 2003, 423, 968], microelectromechanical systems [Brosseau, C.; Ben Youssef, J.; Talbot, P.; Konn, A. M. J. Appl. Phys. 2003, 93, 9243], and others [Pileni, M. P. Adv. Funct. Mater. 2001, 11, 323] was described. These applications are based in their high specific surface area, in their capacity to traverse biological barriers, biocompatibility, ion absorption capacity, and mainly on their exclusive magnetic properties which only appear at nanometric level, such as superparamagnetism, magnetoresistance, magnetic anisotropy, etc.

One of the most important features of these materials is that their properties vary extensively with size [Iglesias, O.; Labarta, A. Phys. Rev. B, 2001, 63, 184416], internal structural disarrangement [Serna, C. J.; Bodker, F.; Morup, S.; Morales, M. P.; Sandiumenge, F.; Veintemillas-Verdaguer, S Solid State Comm. 2001, 118, 437], and aggregation state [Koutani, S.; Gavoille, G.; Gérardin, R. J. Magn. Magn. Matter. 1993, 123, 175]. For example, it is well known in the hyperthermia field that the specific absorption rate (SAR) for a determined alternation frequency and field intensity comes from particles in a very narrow size range.

In magnetic resonance imaging, magnetic nanoparticles work by means of changing the relaxation time in adjacent tissue due to the bipolar magnetic interactions with aqueous protons. The efficiency of a contrast agent in magnetic resonance is measured by relaxivity. Relaxivity is defined as the increase in the proton relaxation rate induced by the contrast agent per concentration unit of the contrast agent. In this case, relaxivity is also related with the particle size and is more homogeneous if the size distribution is narrow.

Another feature determining the magnetic properties of nanoparticles is their shape. For example, one of the terms contributing to the anisotropy energy is anisotropy, such that it is greater in elongated particles than in spherical particles. Therefore, it is desirable to develop methods for producing particles with different shapes, and especially with elongated shapes. As a result, one of the essential requirements for producing magnetic particles optimized as contrast and hyperthermal agents is the control of the size, of the size dispersion and of the shape.

For their use in biomedicine, magnetic nanoparticles must further comply with additional requirements such as water dispersability and biocompatibility.

Methods have been described for producing monodisperse magnetic iron oxide particles with a variable size based on the decomposition of iron coordination compounds in organic solvents in the presence of surfactants consisting of a hydrocarbon chain ending in a polar group. However, these compositions are unstable in aqueous medium. A way of solving this problem consists of the absorption of a second surfactant forming a bilayer around the magnetic nucleus. However, this second surfactant layer is easily desorbed unless it is covalently linked to the first layer. Magnetic nanoparticles coated with stable bilayers are also known [Shen, L.; Stachowiak, A.; Hatton. T. A.; Laibinis, P. E.; Langmuir, 2000, 16, 9907] but they are only stable at a pH greater than 7.4. On the other hand, for their application in biomedicine, a method for preparing nanoparticles which is carried out in aqueous medium is preferable in order to favor subsequent biological functionalization processes. Processes for preparing magnetic nanoparticles in aqueous medium are known [U.S. Pat. No. 4,329,241, Massart]. However, said methods can give rise to aggregation problems and are not very competent in controlling the size and size dispersion.

Another methodology to control the growth and aggregation of iron oxide particles consists of precipitating a polymeric matrix in situ. A great variety of natural polymeric matrices have been used, such as dextran [U.S. Pat. No. 4,452,773, Molday), proteins [U.S. Pat. No. 6,576, 221, Kresse], alginates [Kroll, E, Winnik, F. M.; Ziolo, R. F. Chem. Mater. 1996, 8, 1594]; and synthetic polymers such as functionalized polystyrenes [Ziolo, R. F.; Giannelis, E. P.; Weinstein, B A.; Ohoro, M. P.; Ganguly, B. N.; Mehrotra, V.; Russel, M. W.; Huffinan, D. R. Science, 1992, 257, 219], polypyrrole [Bidan, G.; Jarjayes, O., Fruchart, J. M.; Hannecart, E. Adv Mater, 1994, 6, 152], phenolic polymers [Kommareddi, N. S.; Tata, M; John, V. T.; McPherson, G. L.; Herman, M. F.; Lee, Y. S.; O'Connor, C. J.; Akkara, J. A.; Kaplan, D. L. Chem. Mater. 1996, 8, 801], carboxylic acid polymers [WO05112758, Acad], block copolymers [Sohn, B. H.; Cohen, R. E. Chem. Mater. 1997, 9, 264; Kim, J. Y.; Shin, D. H.; Ryu, J. H.; Choi, G. H.; Suh, K. D. J. Appl. Polym. Sci. 2004, 91, 3549], and others [LesliePelecky, D. L.; Rieke, R. D. Chem. Mater. 1996, 8, 1770]. One of the preferred techniques for increasing the stability of the coating consists of cross-linking polymeric chains [WO03005029, Xu]. However, this methods do not offer the possibility of systematically varying the particle size, they often yield wide size distributions and occasionally, they are not stable in aqueous dispersions.

Another desirable feature for biomedical uses is to prevent the reaction of the immune system against the nanoparticles by means of coatings minimizing said response to achieve higher dwelling times of the nanoparticles in the organism. It is also desirable to anchor to the surface of the particles biologically active molecules allowing a specific localization or a biological functionality. [U.S. Pat. No. 6,514,481, Prasad] describes silica-coated iron oxide nanoparticles to which a peptide is attached by means of a spacer and in [WO 02098364, Perez Manual], the iron oxide nanoparticles are coated with dextran to which peptides and oligonucleotides are anchored.

There is thus a demand for processes for producing biocompatible nanoparticles with a variable size and shape, low size dispersion, which can be stably and homogenously dispersed in physiological media, with coatings which allow them to avoid the attack of the immune system and with functional groups on their surface allowing the anchoring of molecules with a biological functionality. But there is especially a demand for processes which can simultaneously respond to all these demands. The objective of this invention is to respond to this demand.

### BRIEF DESCRIPTION OF THE INVENTION

One aspect of the present invention relates to a system comprising magnetic nanoparticles of a metal oxide comprising iron and a polymer (P) in which:

a) the polymer comprises a monomer (I) containing active functional groups which can interact with metal ions by means of Coulomb forces, Van der Walls forces or coordination bonds

b) the molar [Fe]/[monomer (I)] ratio is 0.01-10,

c) the nanoparticles have a size dispersion of less than 15% of the average size.

According to one variant, the system is solid (nanocomposite) and according to another variant, the system is liquid (ferrofluid).

Another variant of the system comprises a polymer (P) which, apart from monomer (I), comprises a monomer (II) containing hydrophilic functional groups.

According to another variant, the system comprises a polymer (P) which, apart from monomers (I) and (II) comprises a monomer (III) containing functional groups which can anchor active biological molecules.

A second aspect of the present invention relates to a process for obtaining a system comprising magnetic nanoparticles of a metal oxide comprising iron and a polymer (P) as defined, comprising:

a) mixing
a1) an aqueous solution, optionally mixed with organic solvents, of a polymer (P) comprising a monomer (I) containing active functional groups which can intercat with metal ions by means of coulomb forces, Van der Waals forces or coordination bonds, with
a2) an aqueous solution, optionally mixed with organic solvents, comprising at least one Fe salt in which the molar [Fe]/[monomer (I)] ratio is 0.01-10

b) adding a base in a sufficient amount to reach pH 8-14.

A third aspect of the present invention refers to the use of a liquid system as defined previously, comprising magnetic nanoparticles of a metal oxide comprising iron and a polymer (P) as defined, for magnetic refrigeration, magnetic printing, magnetic inks, rotor lubrication, electric transformers, low noise-level solenoids, switches, magnetorheological fluids, magnetically active fibers, reinforced polymeric composites, sealing in vacuum systems, damping systems, loudspeakers, magnetic sensors, actuators, catalysis, metal recovery and water purification, inductors and antennas in communication technology, magnetic shields and microwave absorption, polymer curing, epoxy resin hardening, contact-free heating and biotechnological, veterinary and medical applications.

### DESCRIPTION OF THE DRAWINGS

FIG. 1 shows a transmission electronic microscopy image of a section with a thickness of 40 nm of a maghemite nanocompound prepared according to Example 1 containing 5% of iron.

FIG. 2 shows a transmission electronic microscopy image of a maghemite nanocompound sample prepared according to Example 2 from a [Fe]/[pyridine]=0.40 ratio once it has been ground dispersed in acetone and deposited on a grid.

FIG. 3 shows SAXS curves of a polymer (PVP) sample and a series of maghemite nanocompounds (S1, S2, S3, S4 and S5) prepared according to Example 2 after pressing them into tablets with a thickness of 0.1 mm.

FIG. 4 shows the variation of the particle diameter calculated from SAXS curves by means of adjusting to a Guinier expression, in a series of maghemite compounds prepared according to Example 2.

FIG. 5 shows a transmission electronic microscopy image of a maghemite nanocompound sample in the form of a rod prepared according to Example 3 from a polymer of anionic origin containing 27.8% iron, once it has been ground, dispersed in acetone, and deposited on a grid.

FIG. 6 shows an electronic microscopy image of a maghemite fluid prepared according to Example 4.

FIG. 7 shows the magnetization variation against the field in a series of maghemite compounds prepared according to Example 2. The continuous lines correspond to adjustments to a Langevin expression.

FIG. 8 shows the variation of the out-of-phase ac magnetic susceptibility with temperature, for an alternation frequency of 10 Hz, in a series of maghemite nanocompounds prepared according to Example 2.

FIG. 9 shows the variation of the out-of-phase ac magnetic susceptibility with temperature, for different alternation frequencies, in a maghemite nanocompound prepared from an anionic polymer according to Example 3.

FIG. 10 shows the variation of magnetization against temperature in the ferrofluid prepared in Example 4, immediately after the preparation and a month after the preparation.

### DETAILED DESCRIPTION OF THE INVENTION

The inventors have found a system comprising nanoparticles of a metal oxide, comprising iron and a polymer having a low dispersion of the average particle size, where the shape and the average size of the particles can be selected during the preparation process. Said system can be in the form of a solid (nanocomposite) or a liquid (ferrofluid), being able to be adapted to achieve a good dispersibility in the latter case.

When the system is to be used in biotechnological applications, in veterinary applications and in medicine, it can also be modified to obtain biocompatibility, avoid the attack of the immune system and add functional groups allowing the anchoring of molecules with a biological functionality.

A first aspect of the invention relates to a magnetic nanoparticle system comprising magnetic nanoparticles of a metal oxide, comprising iron, and a polymer (P) donde:

a) the polymer comprises a monomer (1) containing active functional groups which can interact with metal ions by means of Coulomb forces, Van der Waals forces or coordination bonds,

b) the molar [Fe ]/[monomer (I)] ratio is 0.01-10,

c) the nanoparticles have a size dispersion of less than 15% of the average size.

According to an embodiment of the invention, in the nanoparticle system the metal oxide contains Fe⁺² and/or Fe⁺³.

A particular embodiment of the invention is the magnetic nanoparticle system in which the metal oxide, apart from Fe, contains a divalent metal, for example, Co²⁺, Ni²⁺, Mn²⁺, Gd²⁺, Be²⁺, Mg²⁺,Ca²⁺, Ba²⁺.

A more particular embodiment of the invention is the magnetic nanoparticle system in which the metal oxide comprises maghemite (γ-Fe₂O₃).

Another particular embodiment of the invention is the magnetic nanoparticle system in which the metal oxide comprises magnetite (Fe₃O₄).

Another particular embodiment of the invention is the magnetic nanoparticle system in which the metal oxide comprises ferrite MFe₂O₄, M being Co²⁺, Ni²⁺, Mn²⁺, Gd²⁺, Be²⁺, Mg²⁺, Ca²⁺ or Ba²⁺.

A particular embodiment of the invention is the magnetic nanoparticle system in which the metal oxide is barium ferrite (BaFe₂O₄).

Polymer (P) can be an organic polymer or an organic polymer containing inorganic residues such as alkoxy silyl, titanium silyl or others, covalently bound to the polymeric chain (hybrid organic-inorganic polymer).

According to an embodiment of the invention, in the magnetic nanoparticle system polymer (P) is an organic polymer.

According to another embodiment of the invention, in the magnetic nanoparticle system polymer (P) is a hybrid organic-inorganic polymer.

One aspect of the invention comprises a polymer (P) comprising a monomer (I) containing active functional groups which can interact with metal ions by means of Coulomb forces, Van der Waals forces or coordination bonds, for example alcohol, alkoxide, carboxyl, anhydride, phosphate and/or phosphine groups. The functional groups can also be nitrogenated functional groups such as amine, amide, nitrile, azide groups. Other nitrogenated functional groups can be imines and heterocyles such as pyridine, pyrrole, pyrrolidone, pyrimidine, adenine.

Therefore, an embodiment of the invention is the magnetic nanoparticle system in which the monomer (I) contains alcohol, alkoxide, carboxylic, anhydride, phosphate and/or phopshine type functional groups.

Another embodiment of the invention is the magnetic nanoparticle system in which the monomer (I) contains nitrogenated functional groups such as amine, amide, nitrile or azide.

Another embodiment of the invention is the magnetic nanoparticle system in which the monomer (I) contains imines, or heterocycles containing nitrogen such as pyridine, pyrrole, pyrrilodone, pyrimidine, adenine.

A particular embodiment is the magnetic nanoparticle system in which the monomer (I) is a vinyl type monomer. The vinyl monomer is preferably vinylpyridine.

The groups which can interact with metal ions by means of Coulomb forces, Van der Waals forces or coordination bonds comply the function of molding the size and the shape of magnetic particles contained in the system during the synthesis thereof. They also comply the function of coating the particles with the organic polymer.

The inventors have discovered that it is possible to control the size of the magnetic nanoparticles of the magnetic nanoparticle system of the invention by varying the molar [Fe]/[monomer I] ratio during the preparation method. The greater the ratio, the larger the size. In the magnetic nanoparticle systems of the invention, the molar [Fe]/[monomer I] ratio varies between 0.01 and 10, preferably between 0.03 and 2.

In the magnetic nanoparticle systems of the invention, the average size of the nanoparticles of metal oxide comprising iron can be of 1 to 1000 nm, preferably of 1 to 100 nm.

The inventors also discovered that the shape of the particles in the magnetic nanoparticle system of the invention can be controlled by means of the use of polymers prepared by different processes. Polymers synthesized by a radical pathway [Odian G. Principles of Polymerization, Wiley-Interscience, New York, 2004] generate spherical particles (Examples 1 and 2), whereas polymers synthesized by an anionic pathway [Odian G. Principles of Polymerization, Wiley-Interscience, New York, 2004] generate elongated particles (Example 3).

Therefore, a particular embodiment of this invention is formed by the magnetic nanoparticle system in which the particles are spherical and polymer (P) is a polymer obtained by a radical pathway.

Another particular embodiment of this invention is formed by the magnetic nanoparticle system in which the particles are elongated and polymer (P) is obtained by an anionic pathway.

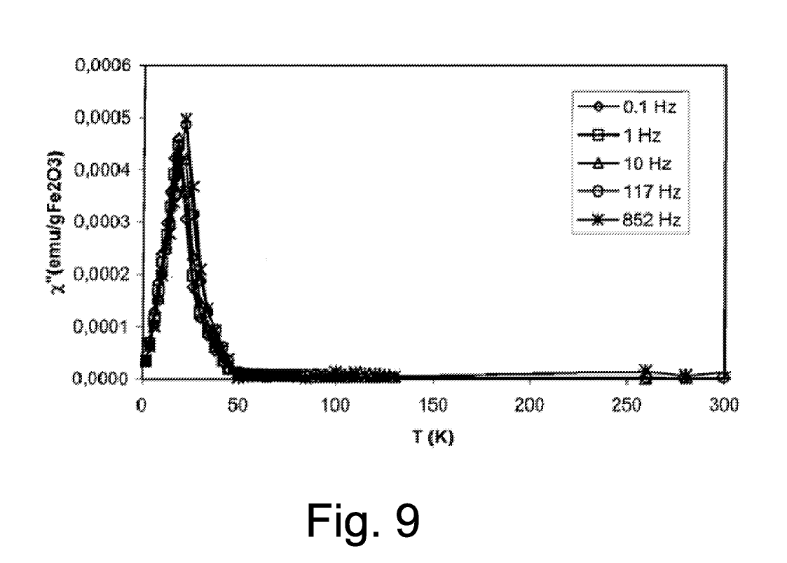

The rod-shaped nanoparticles have an extraordinarily narrow out-of-phase susceptibility peak, as discussed in example 3.2 and shown in FIG. 9. This feature makes said particles be especially suitable for uses in which hyperthermia is a property to be exploited, such as for example in certain oncological treatments of infectious diseases.

The nanoparticle system of the invention can be in solid form or in liquid form.

In the present invention, the magnetic nanoparticle system of the invention in solid form is called “nanocomposite” and the magnetic nanoparticle system of the invention in liquid form is called “ferrofluid”.

As used in the present invention, the term “nanocomposite” relates to dispersions of nanoparticles of a metal oxide comprising iron, in a solid polymer matrix.

A particular aspect of this invention is formed by the solid magnetic nanoparticle system (nanocomposite).

As used in the present invention, the term “ferrofluid” relates to a stable and homogeneous colloidal suspension of magnetic particles, i.e., with a net magnetic moment, in a carrier liquid. The carrier liquid can be, for example, water or an aqueous solution containing a substance acting as a buffer and other water-soluble substances.

Another particular aspect of this invention is formed by the liquid magnetic nanoparticle system liquid (ferrofluid).

A particular embodiment of the invention is the magnetic nanoparticle system liquid (ferrofluid) comprising water or a biocompatible aqueous solution, preferably a biocompatible aqueous solution containing a substance acting as a buffer and optionally other water-soluble substances. In the liquid magnetic nanoparticle system of the invention (ferrofluid), it is important that the iron oxide nanoparticles are homogeneously dispersed in the liquid medium and that the dispersion is stable. In particular, for biotechnological applications, in medicine and in veterinary applications, it is interesting that said dispersion is homogeneous and stable in physiological media and that the nanoparticles are biocompatible. A particular embodiment of the invention is thus the magnetic nanoparticle system in which polymer (P), apart from monomer (I), comprises a monomer (II) containing functional hydrophilic groups.

Another particular embodiment of the invention is the magnetic nanoparticle system in which monomer (II) is a vinyl type monomer, such as acrylate, methacrylate, methyl methacrylate, vinylpyrrolidone and derivatives thereof, preferably polyethylene glycol (PEG) methacrylate.

Another particular embodiment of the invention is the magnetic nanoparticle system in which polymer (P), apart from monomers (I) and (II), comprises a monomer (III) containing functional groups which can anchor biologically active molecules. Said groups can be for example —NH₂; —SH, —COOH, and —CONH₂.

Another particular embodiment of the invention is the magnetic nanoparticle system in which monomer (III) is a vinyl type monomer.

Another particular object of the invention is the magnetic nanoparticle system in which the biologically active molecules are anchored to monomer (III) by means of covalent bonds.

As used herein, the term “biologically active molecules” relates to biological molecules or analogs of biological molecules including a functional group with the capacity to accept electronic density belonging, by way of illustration and without limiting the scope of the present invention, to the following list: amino groups, thiol groups, disulfide groups, dialkyl sulfides, epoxy groups, as well as amines and alcohols in platinum. These biomolecules having said functional groups, both in the structure itself of the molecule and due to the effect of the synthetic addition of said group, can be selected from one of the following groups for example:

a) natural biomolecules: single- or double-stranded nucleic acids (DNA or RNA), enzymes, antibodies, membrane proteins, heat shock proteins, chaperonins, other proteins, monosaccharides, polysaccharides, glycoproteins, fatty acids, terpenes, steroids, other molecules of a lipid nature, lipoproteins, hormones, vitamins, metabolites, hydrocarbons, thiols, or macromolecular aggregates formed by proteins and/or nucleic acids or other combinations of the previously mentioned molecules;

b) natural biomolecules obtained by in vitro selection processes: aptamers, ribozymes, aptazymes; and

c) artificial biomolecules: PNAs, other analogs of natural nucleic acids, natural and artificial nucleic acid chimers, polymers with the capacity to recognize shapes (“molecular imprinted polymers” or MIPs), artificial antibodies, recombinant antibodies and mini-antibodies.

Another particular embodiment of the invention is the magnetic nanoparticle system in which all the monomers in polymer (P) are vinyl type monomers.

A particular embodiment of the invention is the liquid magnetic nanoparticle system (ferrofluid) comprising:

a) maghemite as a metal oxide,

b) a polymer matrix containing:
i. 4-vinylpyridine [monomer (I)],
ii. a vinyl monomer functionalized with poly(ethylene glycol) (PEG) [monomer (II)]
iii. a vinyl monomer containing functional groups selected from —NH2, —SH, —COOH, and —CONH2 [monomer (III)]

c) an aqueous solution of phosphate buffer (PBS), maintaining the system at pH 7.4.

A second aspect of the present invention is formed by the process for preparing the magnetic nanoparticle system comprising the following steps:

a) mixing
a1) an aqueous solution, optionally mixed with organic solvents, of a polymer (P) comprising a monomer (1) containing active functional groups which can interact with metal ions by means of Coulomb forces, Van der Waals forces or coordination bonds, with
a2) an aqueous solution, optionally mixed with organic solvents, comprising at least one Fe salt, in which the molar [Fe]/[monomer (I)] ratio is 0.01-10

b) adding a base in a sufficient amount to reach pH 8-14.

A particular embodiment of the invention is the process for preparing magnetic nanoparticles in which solution a2) comprises at least one salt of a divalent metal and a Fe⁺³ salt. The divalent metal salt can be for example a Fe²⁺, Co²⁺, Ni²⁺, Mn²⁺, Gd²⁺, Be²⁺, Mg²⁺, Ca²⁺ and Ba²⁺.

A more particular embodiment of the invention is the process for preparing magnetic nanoparticles in which the divalent metal salt in solution
a2) comprises a Fe²⁺ salt.

Another more particular embodiment of the invention is the process for preparing magnetic nanoparticles in which in solution a2), the Fe²⁺ salt is FeBr₂ and the Fe⁺³salt is FeBr₃.

Another particular embodiment of the invention is the process for preparing magnetic nanoparticles in which solution a2) further comprises a monovalent bromide, for example KBr, RbBr, NaBr, CsBr, (CH₃)₄NBr, (CH₃CH₂)₄NBr).

Another particular embodiment of the invention is the process for preparing magnetic nanoparticles in which in solution a2), the Fe²⁺ salt is FeCl₂ and the Fe⁺³ salt is FeCl₃.

Another particular embodiment of the invention is the process for preparing magnetic nanoparticles in which solution a2) further comprises a monovalent chloride, for example KCl, RbCl, NaCl, CsCl, (CH₃)₄NCl, (CH₃CH₂)₄NCl).

Another particular embodiment of the invention is the process for preparing magnetic nanoparticles in which solutions a1) and a2) are mixed in a molar [Fe]/[monomer (I)] ratio of 0.01 to 10.

Another particular embodiment of the invention is the process for preparing magnetic nanoparticles in which solutions a1) and a2) are mixed in a molar [Fe]/[monomer (1)] ratio of 0.03 to 2.

Apart from discovering that the molar [Fe]/[monomer] ratio affects the size of the nanoparticles of metal oxide comprising iron, the inventors also discovered that the size of said nanoparticles can be varied by means of using different molar ratios of Fe⁺² and Fe ⁺³ in solution a2).

Therefore, a particular embodiment of the invention is the process for preparing magnetic nanoparticles in which the average size of the nanoparticles of metal oxide comprising iron is regulated by varying the molar ratio of Fe⁺² and Fe⁺³ in solution a2), by means of varying the proportion of the dissolved salts of both cations.

A third discovery of the inventors is that the average size of the nanoparticles of metal comprising iron can be regulated by varying the molar ratio between the base added in b) and the iron contained in a). Therefore, another particular embodiment of the invention is the process for preparing magnetic nanoparticles in which the average size of the nanoparticles of metal oxide comprising iron is regulated by varying the molar ratio between the base added in b) and the iron contained in a).

Another particular embodiment of the invention is the process for preparing magnetic nanoparticles in which in step b) the base is added until reaching a pH of 12.5 to 13.

Another particular embodiment of the invention is the process for preparing magnetic nanoparticles in which the polymer (P) used in a1) comprises a monomer (I) containing active functional groups which can interact with metal ions by means of Coulomb forces, Van der Waals forces or coordination bonds, for example alcohol, alkoxide, carboxyl, anhydride, phosphate, and/or phosphine.

Another particular embodiment of the invention is the process for preparing magnetic nanoparticles in which polymer (P) used in a1) comprises a monomer (I) containing nitrogenated functional groups, such as amine, amide, nitrile, azide groups.

Another particular embodiment of the invention is the process for preparing magnetic nanoparticles in which polymer (P) used in a1) comprises a monomer (I) containing imines or heterocycles such as pyridine, pyrrole, pyrrolidone, pyrimidine, adenine.

Another particular embodiment of the invention is the process for preparing magnetic nanoparticles in which polymer (P) used in a1) comprises a vinyl type monomer (I).

Another particular embodiment of the invention is the process for preparing magnetic nanoparticles in which polymer (P) used in a1) comprises vinylpyridine as a vinyl monomer.

Another particular embodiment of the invention is the process for preparing magnetic nanoparticles in which polymer (P) is obtained by a radical pathway.

Another particular embodiment of the invention is the process for preparing magnetic nanoparticles in which polymer (P) is obtained by an anionic pathway.

The process can include a polymer (P) preparation step. Therefore, another particular object of the invention is the process for preparing magnetic nanoparticles in which polymer (P) is prepared by means of a process previos to step a).

Another particular embodiment of the invention is the process for preparing magnetic nanoparticles in which polymer (P) is a copolymer and is prepared by simultaneous or successive copolymerization of a monomer (I) with a monomer (II) containing hydrophilic groups and optionally with a monomer (III) containing functional groups which can anchor biologically active molecules.

Monomers (II) and (III) are as described previously in this application.

The copolymerization of the polymer P can also be carried out after preparing the magnetic nanoparticles of the invention. Thus, a particular embodiment of the invention is the process for preparing magnetic nanoparticles in which, after step b), the process comprises a step c) comprising the copolymerization of polymer (P) with a monomer (II) containing hydrophilic groups and optionally with a monomer (III) containing functional groups which can anchor biologically active molecules, and when the copolymerization is carried out with the two monomers (II) and (III), said copolymerization is carried out successively or simultaneously.

Another particular embodiment of the invention is the process for preparing magnetic nanoparticles which comprises subjecting the product of step b) to a solid-liquid phase separation to obtain a solid system (nanocomposite) comprising magnetic nanoparticles containing a metal oxide core and a polymer (P).

Another particular embodiment of the invention is the process for preparing magnetic nanoparticles comprising subjecting the product of the additional step c) to a solid-liquid phase separation to obtain a solid system (nanocomposite) comprising magnetic nanoparticles of a metal oxide comprising iron and an organic polymer (P).

An additional step to the process for preparing magnetic nanoparticles of the invention comprises dispersing the solid product (nanocomposite) in a suitable liquid medium to obtain a liquid system (ferrofluid). In a particular embodiment, the liquid is water or a biocompatible aqueous solution, preferably the aqueous solution acting as a buffer.

Thus, a particular embodiment of the invention is the process for preparing magnetic nanoparticles in which, after steps b) or c), the solid product (nanocomposite) is dispersed in a suitable liquid medium to obtain a liquid system (ferrofluid).

Another particular embodiment of the invention is the process for preparing magnetic nanoparticles in which the solid product (nanocomposite) is dispersed in a biocompatible aqueous solution.

Another particular embodiment of the invention is the process for preparing magnetic nanoparticles in which the solid product (nanocomposite) is dispersed in an aqueous solution comprising a substance acting as a buffer.

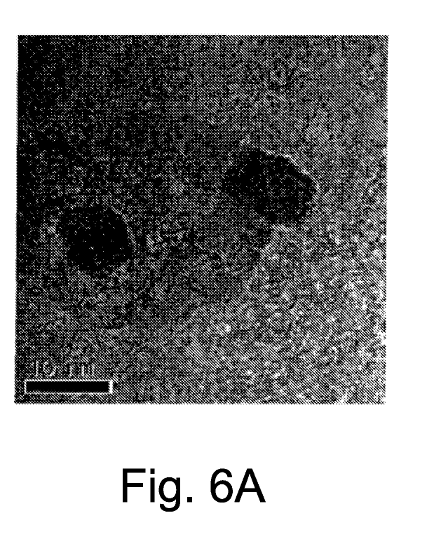

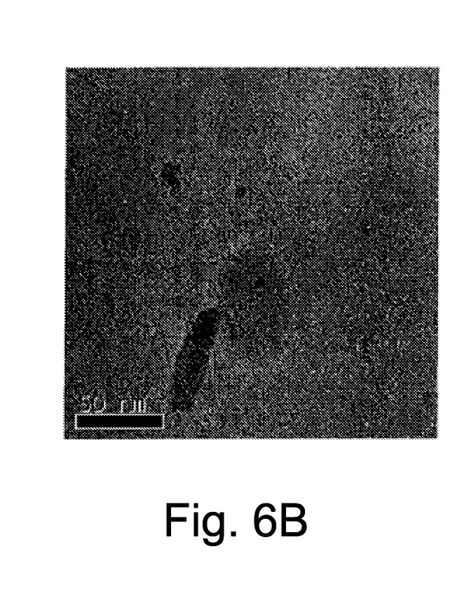

A particularity of liquid nanoparticle systems (ferrofluids) is that the size of the particles is not modified in relation to the size of the particles in the solid system (nanocomposite) and aggregates are not observed, as shown in FIG. 6.

Another aspect of the invention is the use of the ferrofluid of the invention and of the nanoparticles it comprises in industrial applications belonging, for example, to the following group: magnetic refrigeration, magnetic printing, magnetic inks, rotor lubrication, sealing in vacuum systems, damping systems, loudspeakers, magnetic sensors, actuators, catalysis, metal recovery and water purification, inductors and antennas in communication technology, magnetic shields and microwave absorption, biotechnological, veterinary and medical applications [Jech T. J., Odenbach S. Ferrofluids: Magnetically Controllable Fluids and Their Applications, Springer, Berlin, 2002; Goldman A. J. Handbook of Modern Ferromagnetic Materials, Kluwer Academic Publishers, Norwell, 2002]. The industrial applications based on the magnetothermal properties of the magnetic nanoparticles include but are not limited to the hyperthermal use of magnetic nanoparticles in curing polymers, hardening epoxy resins, contact-free heating and biomedical applications.

As mentioned previously, the nanoparticles of the ferrofluid of the invention can anchor biologically active molecules which opens up the biotechnological field of the applications thereof in any of the specific areas, for example, food and agriculture, environment, chemical synthesis by means of enzymes, veterinary applications and medicine. A particular embodiment of the invention is formed by the use of the ferrofluid of the invention in the field of diagnosis and therapeutics of human and animal diseases.

In this sense, the use of this ferrofluid with the nanoparticles in diagnosis and clinical treatment involves a very significant progress in these fields because, for example, a small amount of magnetic nanoparticles can be resuspended in large volumes of sample to be analyzed and later recovered by means of applying an external magnetic field. It is thus possible to purify and/or pre-concentrate very small diluted amounts of a target biological molecule which is specifically hybridized with an organic biomolecule acting immobilized on said nanoparticles, whereby the detection limit is reduced to a great extent and the possibilities of a correct clinical diagnosis are exponentially improved.

This type of systems allows determining the presence of specific biological material of interest in situations in which an early detection thereof can be critical, to prevent the harmful effects entailed by the existence of the species or strains of organisms having said characteristic sequences. This fact has great application in human and veterinary biomedicine, including in the following aspects: i) detection of viral, bacterial, fungal or protozoan type pathogens; ii) characterization of mutations or genetic polymorphisms (SNPs) in said agents which can make them resistant to drugs or facilitate vaccine escape; iii) characterization of mutations or SNPs in human or animal genes related to diseases or prone to them; iv) detection of human disease markers as specific tumors. This detection potential also has important applications in food and environmental control in aspects including the following: i) detection of specific microorganisms, pathogens or contaminants; ii) detection of the presence of genetically modified organisms (GMOs) or transgenic organisms, it being possible to quantify if their presence is above the allowed limits.

On the other hand, these ferrofluids can also be used in human therapy when it is necessary to destroy cells in patients, for example, cancer cells, immune system cells in autoimmune processes, pathogenic microorganisms, etc. Nanoparticles can also have biomolecules, an antibody for example, anchored thereto, which by specifically recognizing a specific tumor marker, a breast cancer marker for example, which allows carrying the nanoparticle to these target cells, which target cells would transfer said nanoparticle to their inside, in which place the target cell could be destroyed thanks to the hyperthermia property.

### EMBODIMENTS

### Example 1

Preparation of a maghemite-poly(4-vinylpyridine) nanocompound/nanocomposite of the invention

1.1. Synthesis of poly(4-vinylpyridine) by a radical pathway.

Approximately 2 grams of the 98% 4-vinyl pyridine monomer previously treated in a molecular sieve were weighed. At the same time a dry schlenk type flask was prepared and immersed in an oil bath at 60° C. The monomer and 15-20 mL of tetrahydrofuran (THF) were introduced in this flask. The mouth of the flask was sealed with a septum and 2 or 3 vacuum-argon cycles were carried out. When the flask is under an argon atmosphere, 2% AlBN was added. It was allowed to react with stirring for 24 hours. Methanol drops were added for the purpose of stopping the reaction.

In order to purify the polymer obtained, it was precipitated by adding cold hexane to the solution and it was subsequently plate-filtered.

1.2. Preparation of a nanocompound of maghemite-poly(4-vinylpyridine). For the preparation of an Fe-polymer compound, 0.2 grams of radical poly(4-vinylpyridine) were first dissolved in 5 mL of a 50% mixture of water and acetone and 0.1 mL of a solution containing 0.11 moles/L of FeBr₂, 0.89 moles/L of FeBr₃ and 0.5 moles/L of RbBr were added. It was evaporated to dryness, first at room temperature and then in an oven at 50° C.

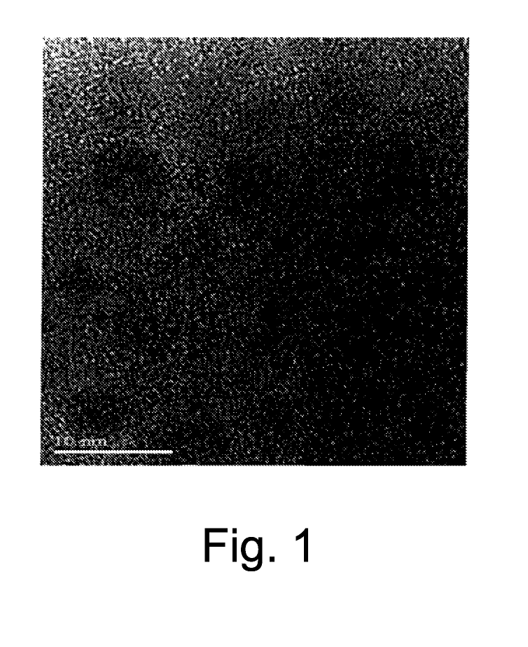

The previously obtained Fe-polymer compound was then immersed in 5 mL of 1 M NaOH for 1 hour. It was filtered and washed with water until the pH of the washing water decreases to 7. It was dried, first at room temperature and then in an oven at 60° C. A nanocomposite was obtained which according to images obtained by high resolution transmission electronic microscopy (HRTEM) (FIG. 1) contains disperse spherical iron oxide nanoparticles with an average size of 4.0 nm and a standard deviation of ±0.4 nm, approximately 10% of its average size, in a solid polymer matrix.

The electronic diffraction analysis of said nanocomposite shows that said nanoparticles have a spinel structure and can therefore consist of maghemite or magnetite. The analysis of the nanoparticles by means of spectroscopy of the energy loss of electrons shows that said particles consist of maghemite (data not shown). The analysis of the nanocompound by titration with K₂Cr₂O₇ indicates the absence of Fe²⁺ ions, which definitively discards the presence of magnetite in the nanocompound.

### Example 2

Preparation of a series of maghemite-poly(4-vinylpyridine) nanocompounds/nanocomposites containing spherical nanoparticles with an average diameter which can vary between 1.5 nm and 15 nm.

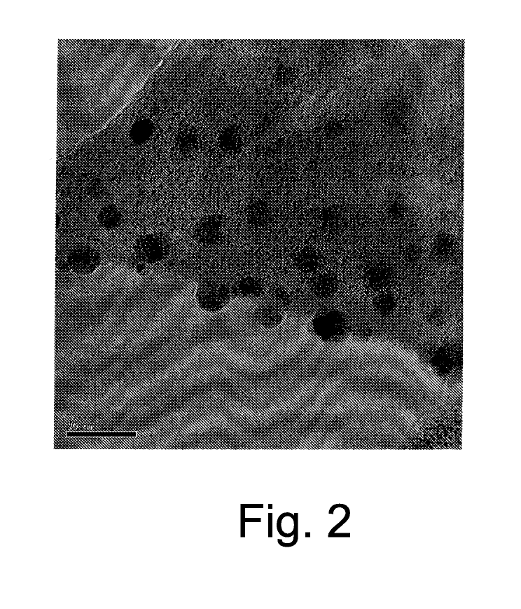

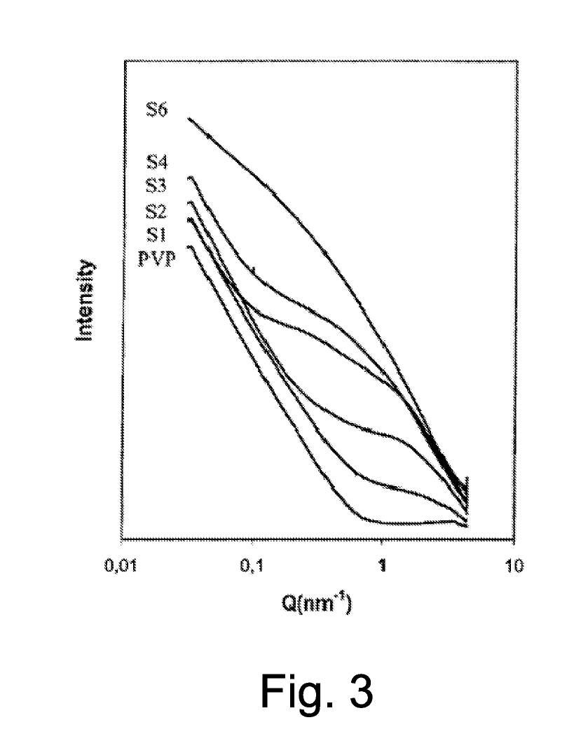

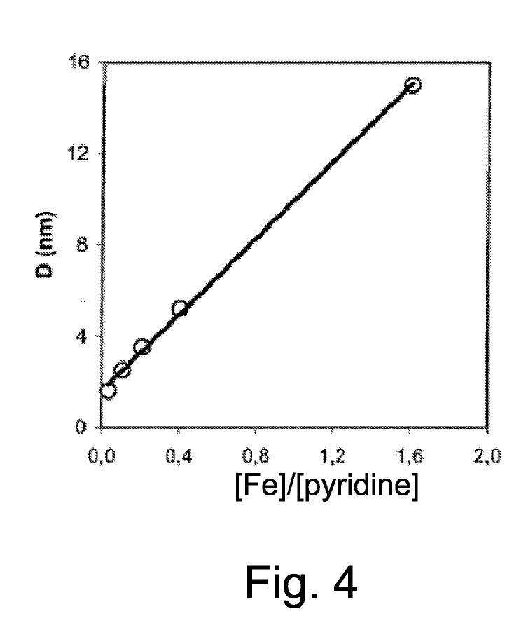

5 type 1 polymer solutions were prepared by means of dissolving 0.4 g of radical poly(vinylpyridine) respectively in 10 mL of a 50% mixture of water and acetone. Amounts of 0.15, 0.88, 1.76, 2.64, 3.52, 6.60 mL respectively of a solution containing 0.40 moles/L of FeBr₂, 0.60 moles/L of FeBr₃ and 0.5 moles/L of RbBr were added. It was evaporated to dryness, first at room temperature and then in an oven at 50° C. Each of the Fe-polymer compounds was immersed in 40 mL of 1 M NaOH respectively for 1 hour. It was filtered and washed with water until the pH of the washing water decreases to 7. It was dried, first at room temperature and then in an oven at 60° C. Six nanoocompounds, called S1, S2, S3, S4, and S5, respectively, were obtained. A study of the size distribution of sample S4 from images obtained by high resolution transmission electronic microscope (HRTEM) (FIG. 2) indicates that the particles are spherical with an average size of 6 nm and a standard deviation of ±0.7 nm. The electro diffraction analysis shows that said particles have a spinel structure and can therefore consist of maghemite or magnetite. The analysis of the particles by titration with K₂Cr₂O₇ indicates the absence of Fe²⁺, which definitively discards the presence of magnetite in the nanocompound. The analysis of nanocompounds S1, S2, S3, S4, S5 by small-angle X-ray scattering (SAXS) (FIG. 3) indicates that the particles are spherical and have an average size of 1.6 nm, 2.5 nm, 3.5 nm, 5.2 nm, 15 nm respectively. It was observed that the variation of the size of the particles with the [Fe]/[pyridine] ratio used in the preparation follows a virtually linear trend (FIG. 4).

### Example 3

Preparation of a maghemite poly(4-vinylpyridine) nanocompound/nanocomposite containing rod-shaped nanoparticles.

3.1. Synthesis of poly (4-vinylpyridine) by an anionic pathway.

Approximately 2 grams of the 98% 4-vinyl pyridine monomer previously treated in a 4^{a} type molecular sieve were weighed. At the same time, a dry schlenk type flask was prepared and immersed in a bath at −78° C. consisting of a mixture of isopropanol and liquid nitrogen. The monomer and 15-20 mL of distilled tetrahydrofuran (THF) were introduced in this flask. The mouth of the flask was sealed with a septum and 2 or 3 vacuum-argon cycles were carried out. When the flask is under an argon atmosphere, 5% BuLi was added. The start of the reaction gives rise to reddish orange color of the solution due to the fact that the carboanion is colored. It was allowed to react with stirring for 24 hours. Methanol drops were added for the purpose of stopping the reaction.

In order to purify the polymer obtained, it was precipitated by adding cold hexane to the solution and it was subsequently plate-filtered.

3.2. Preparation of a rod-shaped of maghemite-poly(4-vinylpyridine) nanocomposite.

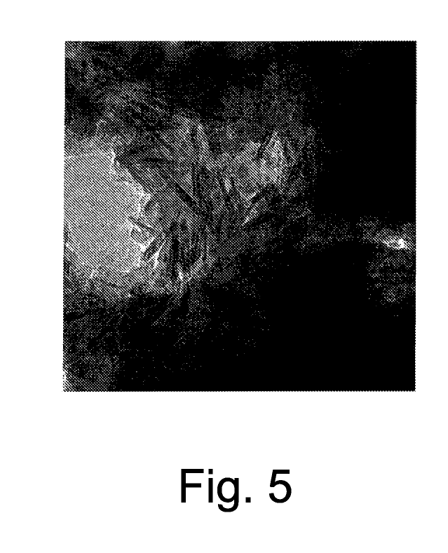

A type 1 polymer solution was prepared by means of dissolving 0.3 g of anionic poly(4-vinylpyridine) in 5 mL of a 50% mixture of water and acetone. 0.506 mL of a solution containing 0.5 moles/L of FeBr₂, 1 mol/L of FeBr₃ and 0.5 moles/L of RbBr were respectively added. It was evaporated to dryness, first at room temperature and then in an oven at 50° C. The Fe-polymer compound obtained was immersed in 20 mL of 1 M NaOH for 1 hour. It was filtered and washed with water until the pH of the washing water decrease to 7. It was dried, first at room temperature and then in an oven at 60° C. The examination of the sample by high resolution transmission electronic microscopy (HRTEM) (FIG. 5) indicates that the particles are rod-shaped with an average length of 60 nm and a thickness of 6 nm. The electron diffraction analysis shows that said particle have a spinel structure and can therefore consist of maghemite or magnetite. The analysis of the particles by titration with K₂Cr₂O₇ indicates the absence of Fe²⁺ ions, which definitively discards the presence of magnetite in the nanocompound.

The analysis by means of small angle neutron scattering (SANS) of a dispersion of the nanocompound in a solution containing 0.1 mol/L of HNO₃ in a 40% mixture of D₂O and H₂O, which cancels the dispersion of the polymer, gives an I(Q) curve corresponding only to the scattering of the particles consists of a line with gradient −2. This result can be interpreted considering a planar particle shape or by means of associating the elongated particles in planar structures in accordance with the observations carried out by HRTEM.

Furthermore, measurements were carried out of the variation of the alternating (ac) susceptibility against the temperature for different field alternance frequencies (FIG. 9) in a nanocompound prepared according to this example containing rod-shaped particles. It was observed that the out-of-phase susceptibility peak is extraordinarily narrow. This quality makes said particles be very suitable for hyperthermal uses, i.e. for heating cells, tissue or non-biological media if they are industrial applications by means of an alternating magnetic field, given that the magnetocaloric effect for a certain field frequency and intensity are generated in a very narrow susceptibility range.

### Example 4

Preparation of a stable maghemite ferrofluid of the invention at pH=7.4 by means of copolymerization with polyethylene glycol methacrylate.

10 milligrams of maghemite-poly(4-vinylpyridine) nanocomposite obtained previously according to Example 1 were dissolved in 1 mL of 0.1 M HNO₃. It was resuspended in 1 mL of phosphate buffer solution (PBS) at pH 7.4, a turbid dispersion being generated.

10 mg of maghemite-poly(4-vinylpyridine) nanocomposite obtained previously according to the Example 1 were dissolved in 1 mL of 0.1 M HNO₃. A solution formed by 1 mL of ferrofluid at pH 2.4 and 3 mL of poly (ethylene glycol) (PEG) functionalized with a methacrylate group with a concentration of 7.5 mg/mL was added. It was resuspended in 1 mL of phosphate buffer solution (PBS) at pH 7.4, a turbid dispersion being originated.

10 mg of nanocomposite of maghemite-poly(4-vinylpyridine) were dissolved in 1 mL of 0.1 M HNO3. A solution formed by 1 mL of ferrofluid at pH 2.4 and 3 mL of poly (ethylene glycol) (PEG) functionalized with a methacrylate group with a concentration of 7.5 mg/mL was added. Said dispersion was incubated at 40° C. for 8 hours to achieve the copolymerization of PEG methacrylate with the polyvinylpyridine coating the nanoparticles. After this incubation process, 300 μL of this mixture were resuspended in 1 mL of phosphate buffer solution (PBS) at pH 7.4 to adjust the acidity to a physiological pH, thus generating the ferrofluid of the invention, which is transparent, stable at physiological pH, biocompatible and can be biologically functionalized.

The obtained dispersion is purified by means of magnetic separation and subsequent re-dispersion in phosphate buffer solution (PBS) at pH 7.4.

The transmission electronic microscopy images of the ferrofluid show that the size of the nanoparticles is not modified with respect to the starting nanocompound and large aggregates are not observed (FIG. 6).

### Example 5

Controlled variation of the magnetic properties in nanocomposites with different particle sizes.

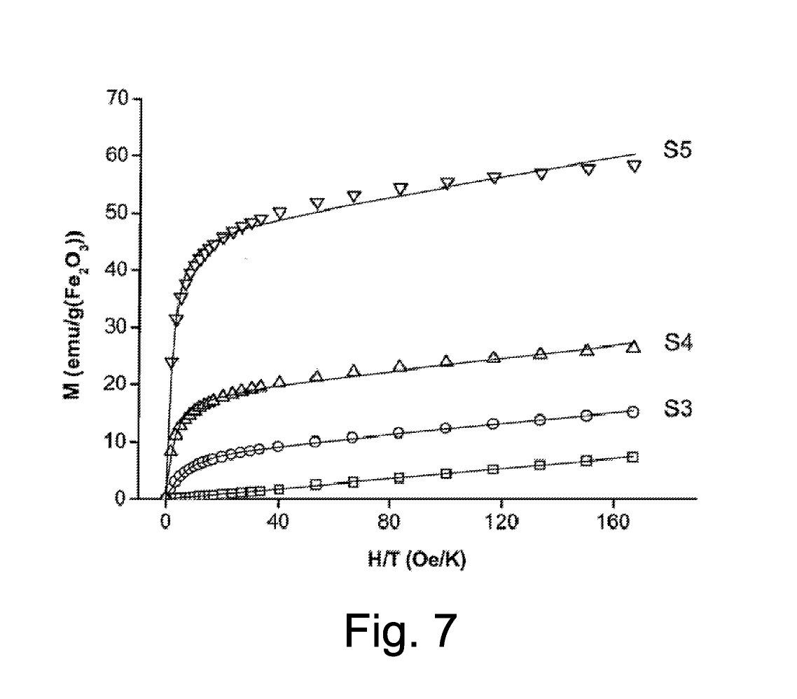

Measurements of the variation of magnetization against the applied field in nanocomposites S1-S5 of Example 2 were carried out. A regular increase was observed in the magnetization curves obtained in nanocompounds with an increasing particle size (FIG. 7). The saturation magnetization of the different nanocomposites calculated by means of extrapolating the linear part of the curve at 0 field shows a variation from virtually 0 emu/g until 50 emu/g, close to the saturation magnetization of macroscopic maghemite (76 emu/g), or in other word, in virtually the entire superparamagnetic range.

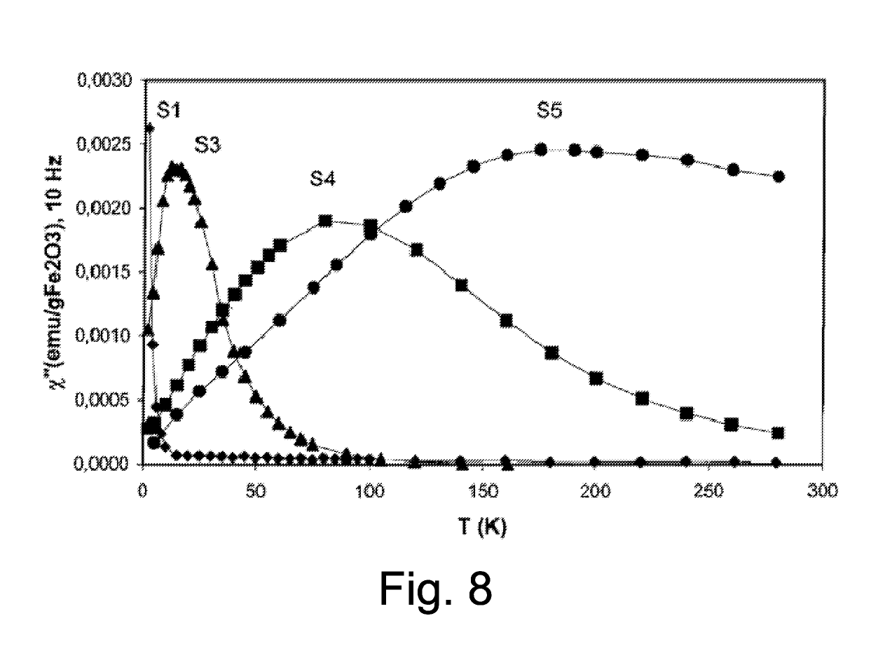

Furthermore, measurements were carried out of the variation of the alternating (ac) susceptibility against the temperature for a field alternation frequency of 10 Hz (FIG. 8) in nanocomposites S1-S5. The blocking temperature of the different nanocompounds, calculated as the susceptibility temperature in the maximum out-of-phase susceptibility, shows a variation from less than 1.8 K to 300 K, or in other words, in virtually the entire superparamagnetic range. Therefore, starting from the basis that the particles are superparamagnetic at room temperature to prevent aggregation, the method allows, within the widest range possible, synthesizing nanoparticles with a maximum magnetocaloric performance for a certain frequency.

### Example 6

Magnetocaloric performance of rod-shaped nanocompounds.

A nanocompound according to this invention containing 28% of rod-shaped particles and 62% of spherical particles is obtained starting from a commercial poly(4-vinylpyridine) supplied by Aldrich and according to the process described in Example 1, but using 1 mL of the FeBr₂/FeBr₃/RbBr solution instead of the amount specified in the example. It was calculated from the images obtained by HRTEM that the rod-shaped particles have an average length of 18.4 nm and an average thickness of 2.7 nm and that the spherical particles have an average diameter of 6.2 nm. The magnetocaloric performance of this nanocompound in an aqueous suspension based on the relative temperature increase was measured in the presence of an alternating magnetic field with an intensity of and an alternation frequency of 144 Hz. A SAR performance=144 w/g is obtained. The magnetocaloric performance of a nanocompound prepared according to this invention starting from a radical polymer containing spherical maghemite nanoparticles with an average size of 7.5 nm was measured in the same conditions. A SAR yield=8 w/g was obtained.

### Example 7

Stability of the magnetic ferrofluids in physiological media.

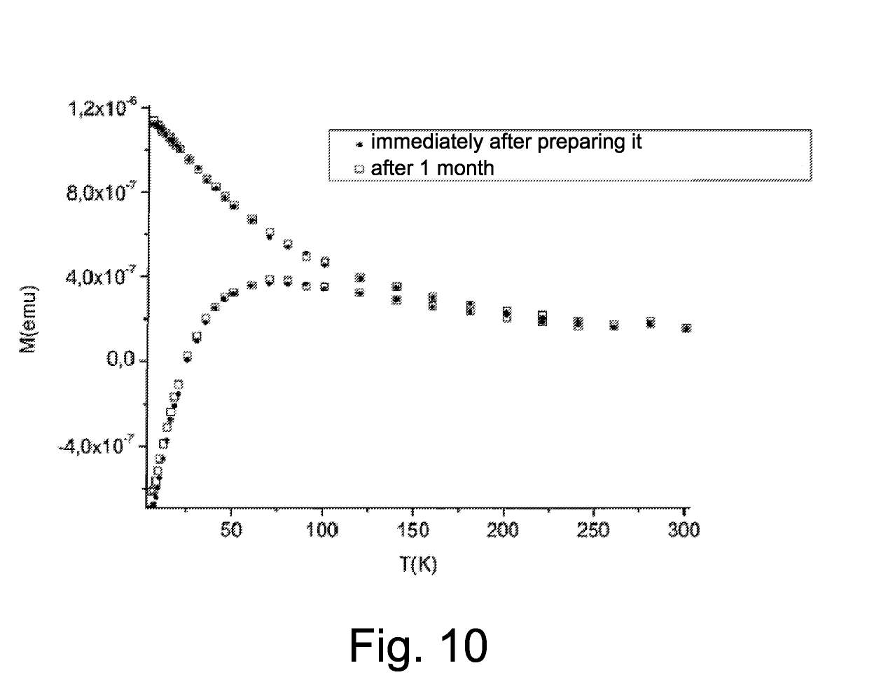

The variation of the magnetization against the temperature with cooling at 0 field and with cooling with a 25 Gauss field (ZFC-FC) was measured in the ferrofluid prepared in Example 4, immediately after the preparation and a month after the preparation. The results show that the curves are perfectly superimposed (FIG. 10), indicating that during this time, the average particle size has not been modified and aggregates have not been formed.

1. A magnetic nanoparticle system comprising magnetic nanoparticles of a metal oxide comprising Fe and-a polymer (P) wherein:
a) polymer (P) comprises:
a vinyl type monomer (I) containing: (i) oxygenated functional groups selected from alcohol, alkoxide, carboxyl, anhydride, phosphate, and phosphine; or (ii) nitrogenated functional groups selected from amine, amide, nitrile, azide, imine, and heterocycles; and
a vinyl type monomer (II) containing hydrophilic groups selected from acrylate, methacrylate, methyl methacrylate, vinylpyrrolidone, and derivatives thereof;
wherein monomer (I) and (II) are different from each other;
b) the molar [Fe ]/[monomer (I)] ratio is 0.01 to 10;
c) the nanoparticles have a size dispersion of less than 15% of the average size; and
d) the nanoparticles have an average size of 1 to 100 nm.

2-5. (canceled)

6. A The magnetic nanoparticle system according to claim 1, wherein the metal oxide comprises maghemite (γ-Fe₂O₃), magnetite (Fe₃O₄), or a metal oxide MFe₂O₄ (ferrite), wherein M is selected from Co²⁺, Ni²⁺, Mn²⁺, Gd²⁺, Be²⁺, Mg²⁺, Ca²⁺, and Ba²⁺.

7-12. (canceled)

13. The magnetic nanoparticle system according to claim 1, wherein monomer (I) contains nitrogenated heterocycle groups selected from pyridine, pyrrole, pyrrolidone, pyrimidine, and adenine.

14-25. (canceled)

26. The magnetic nanoparticle system according to claim 1, wherein monomer (II) is polyethylene glycol.

27. The magnetic nanoparticle system according to claim 1, wherein polymer (P) further comprises a vinyl type monomer (III) containing functional groups which can anchor biologically active molecules.

28. (canceled)

29. The magnetic nanoparticle system according to claim 28, wherein monomer (III) contains functional groups selected from NH₂, —SH, —COOH, and —CONH₂.

30. (canceled)

31. (canceled)

32. A liquid magnetic nanoparticle system (ferrofluid) comprising
a) maghemite as a metal oxide; and
b) a polymer matrix comprising:
4-vinylpyridine [monomer (I)],
a vinyl monomer (II) functionalized with poly(ethylene glycol) (PEG),
a vinyl monomer (III) containing functional groups selected from NH₂, —SH, —COOH, and CONH₂, and
an aqueous solution of phosphate buffer (PBS), maintaining the system at pH 7.4.

33. A process for preparing a magnetic nanoparticle system comprising the following steps:
a) mixing
an aqueous solution (a1), optionally mixed with organic solvents, comprising a polymer (P), wherein polymer (P) comprises a vinyl monomer (I) containing: (i) oxygenated functional groups selected from alcohol, alkoxide, carboxyl, anhydride, phosphate, and phosphine; or (ii) nitrogenated functional groups selected from amine, amide, nitrile, azide, imine, and heterocycles; with an aqueous solution (a2), optionally mixed with organic solvents, comprising at least one Fe salt in which the molar [Fe]/[monomer (I)] ratio is 0.01 to 10;
b) adding a base in a sufficient amount to reach pH 8 to 14; and
c) copolymerizing with vinyl type monomer (III) containing hydrophilic groups selected from acrylate, methacrylate, methyl methacrylate, vinylpyrrolidone, and derivatives thereof.

34. (canceled)

35. The process according to claim 33, wherein solution (a2) comprises an Fe³⁺ salt and at least one the divalent metal salt selected from Fe²⁺, Co²⁺, Ni²⁺, Mn²⁺, Gd²⁺, Be²⁺, Mg²⁺Ba²⁺, salts.

36. (canceled)

37. The process according to claim 33, wherein solution (a2) contains comprises FeBr₂ and FeBr₃.

38-43. (canceled)

44. The process according to claim 33, wherein polymer the vinyl type monomer (I) contains: (i) oxygenated functional groups selected from alcohol, alkoxide, carboxyl, anhydride, phosphate and/or phosphine; or (ii) nitrogenated functional groups selected from amine, amide, nitrile, azide, imine, pyridine, pyrrole, pyrrolidone, pyrimidine, and adenine.

45-58. (canceled)

59. A method of using the liquid magnetic nanoparticle system (ferrofluid) of claim 32, wherein the use is selected from the group consisting of magnetic refrigeration, magnetic printing, magnetic inks, rotor lubrication, electric transformers, low noise-level solenoids, switchers, magnetorheological fluids, magnetically active fibers, reinforced polymeric composites, sealing in vacuum systems, damping systems, loudspeakers, magnetic sensors, actuators, catalysis, metal recovery and water purification, inductors and antennas in communication technology, magnetic shields and microwave absorption, polymer curing, epoxy resin hardening, contact-free heating and biotechnological, veterinary and medical applications.

60. The method of claim 59, wherein the medical applications comprise application the diagnosis and treatment of human diseases.

61. The process according to claim 33, further comprising the step of copolymerizing polymer P with a monomer (III) containing functional groups which can anchor biologically active molecules and where the copolymerization is carried out with monomers (II) and (III), successively or simultaneously.
